(19) United States
(12) Patent Application Publication
(10) Pub. No.: US 2024/0177315 A1
(43) Pub. Date: May 30, 2024

(54) MEDICAL INFORMATION PROCESSING APPARATUS AND MEDICAL INFORMATION PROCESSING METHOD

(71) Applicant: Canon Medical Systems Corporation, Otawara-shi (JP)

(72) Inventors: Guang Yi ONG, Nasushiobara (JP); Maiko TEZUKA, Nasushiobara (JP); Kenji MATSUE, Nasushiobara (JP)

(73) Assignee: Canon Medical Systems Corporation, Otawara-shi (JP)

(21) Appl. No.: 18/515,362

(22) Filed: Nov. 21, 2023

(30) Foreign Application Priority Data
Nov. 30, 2022 (JP) 2022-191787

(51) Int. Cl. G06T 7/00; G16H 30/40

(52) U.S. Cl. CPC G06T 7/0016; G16H 30/40; G06T 2200/24; G06T 2207/20101; G06T 2207/30096

(57) ABSTRACT

According to one embodiment, a medical information processing apparatus includes processing circuitry. The processing circuitry is configured to acquire a medical image. The processing circuitry is configured to narrow down a plurality of anatomical landmarks each indicating an anatomical position in the medical image to one or more anatomical landmarks to be displayed. The processing circuitry is configured to cause a display to display the medical image on which the one or more narrowed-down anatomical landmarks are superimposed.

### CROSS-REFERENCE TO RELATED APPLICATION

This application is based upon and claims the benefit of priority from the prior Japanese Patent Application No. 2022-191787, filed Nov. 30, 2022, the entire contents of which are incorporated herein by reference.

### FIELD

Embodiments described herein relate generally to a medical information processing apparatus and a medical information processing method.

### BACKGROUND

In recent years, to specify a site of a subject imaged in a medical image, a technique of detecting the site of the subject from the medical image together with its anatomical position, and appending an anatomical landmark indicating the site to the detected anatomical position has been widely employed. The anatomical position of the site of the subject is detected for each of a plurality of local structures set with respect to various bones, muscles, organs, etc. configuring a human body. In accordance therewith, a plurality of anatomical landmarks are displayed in the medical image.

While such a technique poses no particular problems, the present inventors have found, upon investigation, that there is room for improvement in that a large number of anatomical landmarks displayed in the medical image hinder smooth reading of the medical image.

### BRIEF DESCRIPTION OF THE DRAWINGS

FIG. 1 is a conceptual diagram of an in-hospital system including a medical information processing apparatus according to a first embodiment.

FIG. 2 is a block diagram showing a configuration of a medical information processing apparatus according to the first embodiment.

FIG. 3 is a schematic diagram showing an example of association information according to the first embodiment.

FIG. 4 is a flowchart for illustrating an operation according to the first embodiment.

FIG. 5 is a diagram showing an example of an ALD selection screen for illustrating an operation according to the first embodiment.

FIG. 6 is a diagram showing an example of anatomical landmarks in a medical image for illustrating an operation according to the first embodiment.

FIG. 7 is a schematic diagram showing an example of preferential reading information according to a second embodiment.

FIG. 8 is a flowchart for illustrating an operation at step ST60 according to a third embodiment.

FIG. 9 is a schematic diagram showing information selected and stored to be displayed according to a fourth embodiment.

FIG. 10 is a schematic diagram showing an example of observation assistance information according to a fifth embodiment.

FIG. 11 is a schematic diagram showing an example of operation assistance information according to a sixth embodiment.

FIG. 12 is a flowchart for illustrating an operation according to a seventh embodiment.

FIG. 13 is a flowchart for illustrating an operation according to an eighth embodiment.

FIG. 14 is a schematic diagram showing an example of a display screen for illustrating an operation according to the eighth embodiment.

### DETAILED DESCRIPTION

In general, according to one embodiment, a medical information processing apparatus includes processing circuitry. The processing circuitry is configured to acquire a medical image. The processing circuitry is configured to narrow down a plurality of anatomical landmarks each indicating an anatomical position in the medical image to one or more anatomical landmarks to be displayed. The processing circuitry is configured to cause a display to display the medical image on which the one or more narrowed-down anatomical landmarks are superimposed.

Hereinafter, a medical information processing apparatus and a medical information processing method according to the present embodiment will be described with reference to the accompanying drawings. In the embodiments to be described below, elements assigned the same reference symbols are assumed to perform similar operations, and redundant descriptions will be omitted where unnecessary.

### First Embodiment

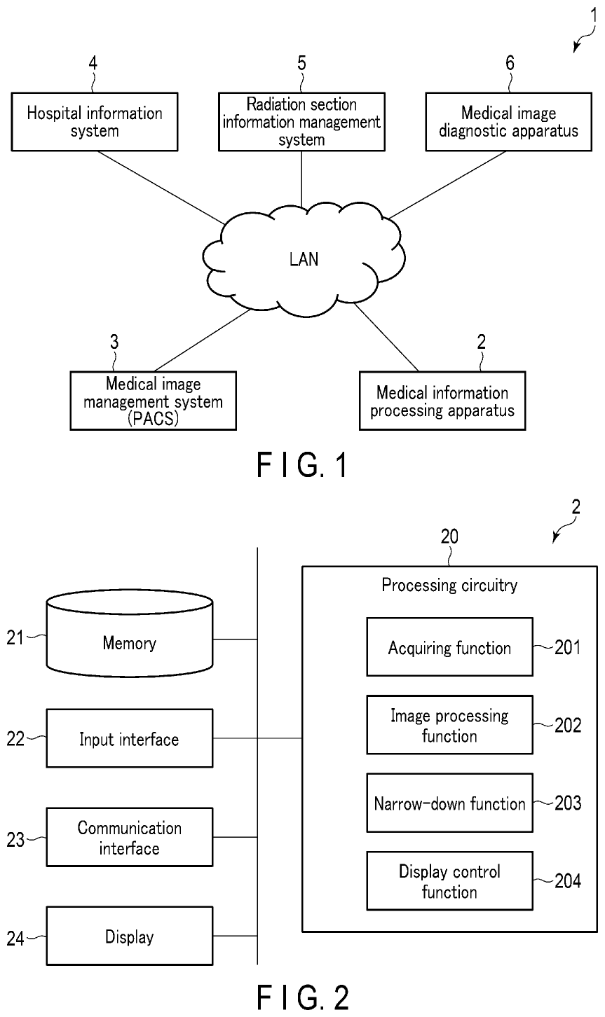

FIG. 1 is a conceptual diagram indicating an in-hospital system including a medical information processing apparatus according to the present embodiment. The medical information processing apparatus 2 in the in-hospital system 1 is connected, via a network, to a medical information management system such as a medical image management system (Picture Archiving and Communication System; PACS) 3, a hospital information system 4, a radiation section information management system (Radiology Information system; RIS) 5, a medical image diagnostic apparatus 6, etc., and exchanges various data with them. It is to be noted that the network may be connected either in a wired or wireless manner. Also, a line via which connection is made is not limited to an intra-hospital network, and may be any line that secures security. For example, connection may be made to a public communication line such as the Internet via a Virtual Private Network (VPN), etc.

The medical image management system 3 is a system that stores medical image data and manages the stored medical image data. The medical image management system 3 stores and manages, for example, medical image data converted in accordance with, for example, Digital Imaging and Communication Medicine (DICOM) standards.

The hospital information system 4 is, for example, a system configured to store intra-hospital information such as diagnosis and treatment information, patient information, and order information, and manage the stored intra-hospital information.

The radiation section information management system 5 is a system configured to manage information such as reservation of an examination in a radiation section, which is one of the diagnosis and treatment sections. Upon receiving, for example, order information and patient information sent from the hospital information system 4, the radiation section information management system 5 sets examination order information in the radiation section.

The medical image diagnostic apparatus 6 is an apparatus configured to generate medical image data by imaging a subject. The medical image diagnostic apparatus 6 generates medical image data by, for example, imaging a patient based on examination order information and patient information managed by the radiation section information management system 5. The generated medical image data is sent to the medical image management system 3 and managed.

Next, the medical information processing apparatus 2 according to the first embodiment will be described with reference to the block diagram of FIG. 2. The medical information processing apparatus 2 is used for, for example, reading a medical image at the time of creating an image diagnostic report (also referred to as a “reading report”).

The medical information processing apparatus 2 according to the present embodiment includes processing circuitry 20, a memory 21, an input interface 22, a communication interface 23, and a display 24. The processing circuitry 20, the memory 21, the input interface 22, the communication interface 23, and the display 24 are connected via a bus, for example, to enable communications therebetween.

The processing circuitry 20 controls the entire operation of the medical information processing apparatus 2 according to an electric signal of an input operation output from the input interface 22. As hardware resources, the processing circuitry 20 includes, for example, a processor such as a CPU, an MPU, and a graphics processing unit (GPU), as well as a memory such as a ROM and a RAM. The processing circuitry 20 executes, with a processor that executes programs expanded in the memory 21, an acquiring function 201, an image processing function 202, a narrow-down function 203, a display control function 204, etc. It is to be noted that the functions 201 to 204 are not necessarily implemented by a single component of processing circuitry. Processing circuitry may be configured by a combination of a plurality of independent processors configured to execute the respective programs to realize the functions 201 to 204. The processes taken charge of by the respective functions to be described below are given for convenience, and may be suitably varied. This is because no matter whether a process is taken charge of by a certain function or taken charge of by another function, the process is executed by the processing circuitry 20 on the whole. The processes taken charge of by the respective functions can be similarly varied in the embodiments and the modifications to be described below.

The acquiring function 201 acquires a medical image from the medical image management system 3 or the memory 21. It is to be noted that the acquiring function 201 and the processing circuitry 20 are examples of an acquisition unit.

The image processing function 202 performs image processing on a medical image. For the image processing, a process of, for example, detecting anatomical positions indicating characteristic local structures of a human body from a medical image, or a process of displaying all the anatomical landmarks (ALDs) corresponding to the detected anatomical positions, for example, may be suitably employed. It is to be noted that a local structure may be referred to as an “ALD”. “ALD” stands for “anatomical landmark”. The detected anatomical positions may be stored in association with the medical image in a data format such as XML data and binary data. Alternatively, the anatomical positions may be stored as incidental information of the medical image according to the DICOM standards. For the process of detecting the anatomical positions, a method that uses a model, for example, may be employed. This type of model is generated by, for example, general machine learning or pattern recognition. For example, models for subjects with different body shapes can be generated using an image database, which is a set of volume data acquired by X-ray CT or MRI, and anatomical position correct data. It is to be noted that the image database includes volume data obtained by imaging a part of a body, as well as whole-body volume data. The anatomical position correct data is data for which the correct anatomical positions of each of the images in the image database have been determined in advance by a specialist such as a doctor. A model is generated by a training algorithm in which features extracted from each item of volume data in the image database and anatomical position correct data are associated with one another. A model shows a method for associating the features extracted from the image database with the anatomical positions. Examples of the model include a trained model using machine learning, etc. Different models may be generated according to sex, age, race, body type, etc.; alternatively, a model that absorbs such a difference may be generated. The detection process is performed by extracting features from the medical image for which anatomical positions are unknown, and detecting the anatomical positions using the generated model. More specifically, after detecting local structures, positions of the detected local structures in the medical image are calculated as the anatomical positions. The calculated anatomical positions are appended to the medical image. The anatomical positions are not necessarily detected by the above-described method, and may be detected by a mathematical statistic framework (a computational anatomical model) known as computational anatomy. For the image processing, a process of, for example, detecting a silhouette of an organ from a medical image and performing measurement from measurement points on the silhouette may be adopted. As an example of this type of measurement, a process is known in which a cardiac silhouette and a thoracic silhouette are detected from a frontal chest X-ray image, a cardiac width and a thoracic width are measured from measurement points on the respective silhouettes, and a cardiothoracic ratio indicating a ratio of the cardiac width to the thoracic width in percentages is calculated. For the image processing, a process of, for example, generating a difference image indicating one or more differences between a medical image and a past medical image corresponding to the medical image may be used.

The narrow-down function 203 narrows down all the anatomical landmarks indicating anatomical positions in the medical image to those to be displayed. It is to be noted that the narrow-down function 203 and the processing circuitry 20 are examples of a narrow-down unit.

The display control function 204 controls, for example, the display 24 so as to display a medical image to be read. The display control function 204 causes the display 24 to display a medical image on which the narrowed-down anatomical landmarks are superimposed. The display control function 204 and the processing circuitry 20 are examples of a display control unit.

It is to be noted that the functions 201 to 204 are not necessarily implemented by a single component of processing circuitry. Processing circuitry may be configured by a combination of a plurality of independent processors configured to execute the respective programs to realize the functions 201 to 204. Alternatively, the functions 201 to 204 may be stored in the memory 21, etc. as programs so that the processing circuitry 20 executes the programs to realize the functions corresponding to the programs.

The memory 21 is a storage device configured to store a variety of information, such as a read-only memory (ROM), a random access memory (RAM), a hard disk drive (HDD), a solid-state drive (SSD), an integrated circuit memory device, etc. The memory 21 may also be a drive or the like that reads and writes a variety of information from and to a portable storage medium such as a CD-ROM drive, a DVD drive, or a flash memory. It is to be noted that the memory 21 is not necessarily realized by a single storage device. For example, the memory 21 may be realized by a plurality of storage devices. Also, the memory 21 may be located within a separate computer connected to the medical information processing apparatus 2 via a network.

The memory 21 stores various programs such as medical information processing programs of the medical information processing apparatus 2, various types of information used in processing, data being processed, and data having been processed. It is to be noted that such programs may be stored in advance in the memory 21. Alternatively, the programs may be stored in a non-transitory computer-readable storage medium and distributed, read from a non-transitory computer-readable storage medium, and installed in the memory 21.

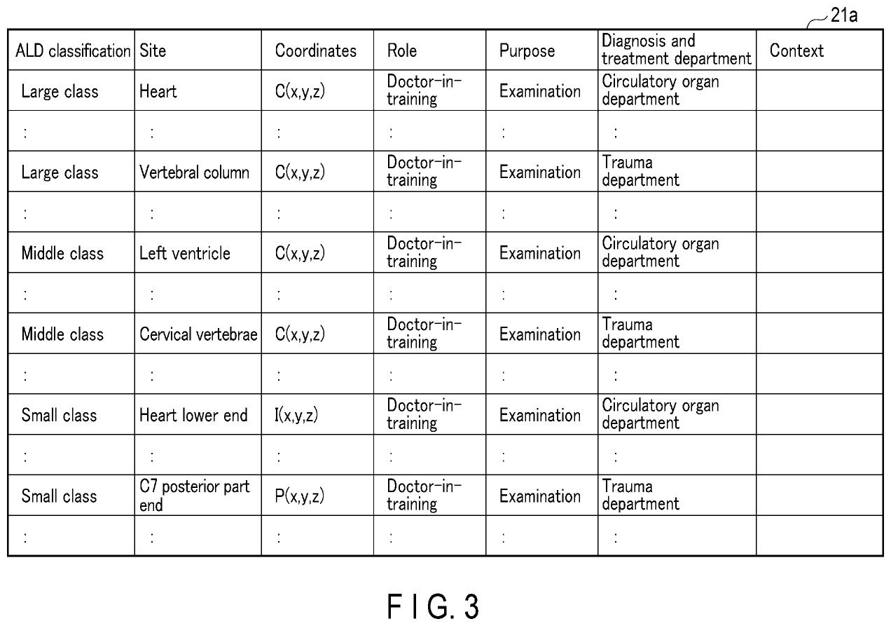

Examples of the various types of information include association information shown in FIG. 3. The association information is, for example, information in which items such as an ALD classification, a site of a subject, coordinates of an ALD, a role, a purpose, a diagnosis and treatment department, and a context are associated. It is to be noted that an anatomical landmark may be suitably referred to as an “ALD” for conciseness of description.

The “ALD classification” indicates one of a large class, a middle class, and a small class, into which the ALDs are classified. The large class covers major ALDs indicating only central points of large organs and bones. The middle class covers secondary ALDs indicating only central points of organs and bones other than the central points covered by the large class. The small class covers tertiary ALDs indicating positions (e.g., anterior, posterior, right, left, superior, and inferior) of organs and bones. It is to be noted that coordinates of a central point can be calculated from coordinates of a three-dimensional medical image. Assuming, for example, that coordinates of a central point are C (x, y, z), coordinates of a central point between a left point L and a right point R are obtained by the following formula:

C(x,y,z)=0.5*[L(x,y,z)+R(x,y,z)]

Similarly, coordinates of a central point between an anterior point A and a posterior point P are obtained by the following formula:

C(x,y,z)=0.5*[A(x,y,z)+P(x,y,z)]

Similarly, coordinates of a central point between a superior point S and an inferior point I are obtained by the following formula:

C(x,y,z)=0.5*[S(x,y,z)+I(x,y,z)]

The “site” indicates a region into which an organ or a bone of a subject is anatomically categorized. Specifically, the “site” indicates a region such as a chest or an abdomen to which a local structure corresponding to the anatomical position belongs.

The “coordinates” indicate an anatomical position of the subject in the medical image. For example, a subject coordinate system (a patient coordinate system) is a coordinate system in which a lateral direction of the subject is taken as an x-axis, a dorsabdominal direction of the subject is taken as a y-axis, and a superior-inferior direction of the subject is taken as a z-axis. The x-axis coordinate increases with the right direction relative to the center of the subject taken as positive, the y-axis coordinate increases with the dorsal direction relative to the center of the subject taken as positive, and the z-axis coordinate increases toward the cranial direction from the caudal direction. Such a subject coordinate system is relatively expressed based on given positions such as reference positions included in the volume data. The subject coordinate system shows an anatomical position based on the x-axis, y-axis, and z-axis coordinates.

The “role” indicates an attribute related to a proficiency level of image reading, such as “doctor-in-training” and “radiology department” (a cancer specialist, a gastrointestinal specialist, or a gynecological specialist). That is, the “role” allows narrowing down to be performed in such a manner that the ALDs to be displayed are suppressed from being eliminated in the case of a doctor-in-training who is inexperienced, the ALDs to be displayed are reduced in number in the case where a doctor (an ordinary doctor) other than a doctor-in-training is to read a site which the doctor is skilled at image reading, and ALDs to be displayed are increased in number in the case where an ordinary doctor is to read a site which the doctor is not skilled at image reading.

The “purpose” indicates a purpose of imaging of the medical image, such as an examination purpose, a measurement purpose, a treatment plan, an explanation for a patient, etc. ALDs that need to be displayed vary according to the purpose (whether it is for hypertension treatment or for cancer follow-up care, for example).

The “diagnosis and treatment department” indicates a diagnosis and treatment department to which a user who is to read the medical image belongs. ALDs that need to be displayed vary according to the diagnosis and treatment department.

The “context” indicates an image reading operation situation (an operation and a situation) on a Bone Subtraction application.

It is to be noted that the various types of information are not limited to the aforementioned ones, and may be suitably associated with an identifier, a name, a reliability, a body tissue, etc. The “identifier” denotes an ID for uniquely specifying an anatomical position. The “name” indicates the name of a local structure using terminology in the anatomical and medical fields. Example names of local structures of the head and the neck include the anterior arch (tubercle) of the atlas (C1), the upper end of the dens (C2), the upper surface of the right eyeball, the upper surface of the left eyeball, the center of the right eyeball, and the center of the left eyeball. Example names of local structures of the chest include the carina of trachea, the apex of the right lung, the apex of the left lung, the inferior angle of the right scapula, the inferior angle of the left scapula, and the origin of the left subclavian artery. Example names of local structures of the abdomen include the superior pole of the right kidney, the superior pole of the left kidney, the inferior pole of the right kidney, the inferior pole of the left kidney, the head of the pancreas, and the tail end of the pancreas. Example names of local structures of the lower limbs include the lateral epicondyle of the right femur, the medial epicondyle of the right femur, the lateral epicondyle of the left femur, the medial epicondyle of the left femur, the lateral condyle of the right tibia, and the medial condyle of the right tibia. The local structures are defined throughout the whole body with the aforementioned granularity, and a plurality of local structures are set with respect to each of various bones, muscles, organs, etc. configuring a human body. An anatomical position is detected for each of the local structures. The “reliability” denotes a numerical value denoting an accuracy of the anatomical position. Since an anatomical position is data estimated by calculation based on a machine learning algorithm, pattern recognition, etc., a numerical value indicating an accuracy with which the position has been calculated is given for each anatomical position. The reliability is expressed in, for example, numerical values from 0 to 1, showing that the closer the numerical value is to 1, the higher the reliability. The “body tissue” denotes a classification made according to the function of the local structure, and is, for example, the nervous system, the osseous system, the respiratory system, or the like.

The input interface 22 receives various input operations from a medical-staff user represented by a doctor, converts the received input operation into an electric signal, and outputs it to the processing circuitry 20. The input interface 22 according to the present embodiment is coupled to an input device such as a microphone, a mouse, a keyboard, a track ball, switches, buttons, a joystick, a touch pad, and a touch panel to which an instruction is input via a touch on its operation screen. It is to be noted that the input device connected to the input interface may be provided on a separate computer connected via a network, etc.

The communication interface 23 performs data communications with the medical image management system 3, the hospital information system 4, the radiation section information management system 5, the medical image diagnostic apparatus 6, etc. The communication interface 23 performs data communications in compliance with, for example, known standards set in advance. Communications with the hospital information system 4 are performed in compliance with, for example, the Health Level 7 (HL7) standards. Communications with the medical image management system 3 are performed in compliance with, for example, the DICOM standards.

The display 24 displays various information in accordance with an instruction from the processing circuitry 20. The display 24 may display a graphical user interface (GUI), etc. for receiving various operations from the user. For the display 24, a cathode ray tube (CRT) display, a liquid crystal display, an organic EL display, an LED display, a plasma display, or any other display may be suitably utilized. It is to be noted that a GUI may be displayed on a display external to a medical information processing apparatus 2 not including a display 24, or a GUI may be displayed via a projector, etc.

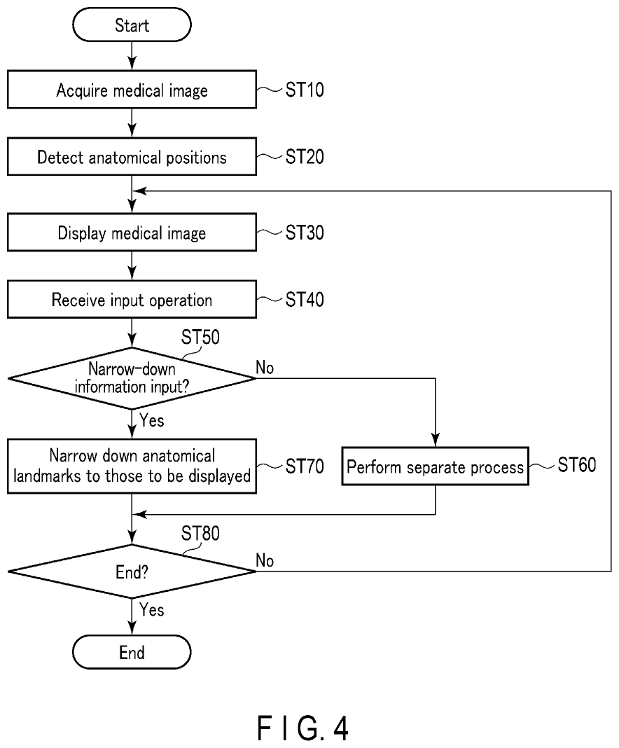

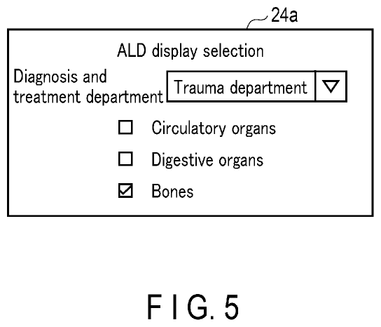

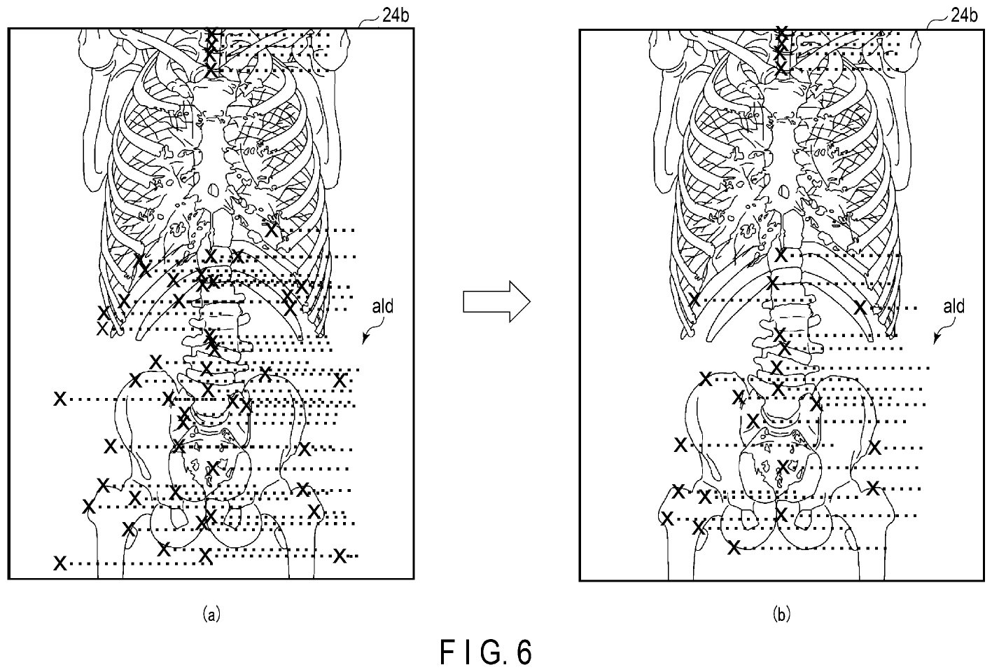

Next, an operation of the medical information processing apparatus with the above-described configuration will be described using a flowchart shown in FIG. 4 and schematic diagrams shown in FIGS. 5 to 6.

At step ST10, the processing circuitry 20 of the medical information processing apparatus 2 acquires a medical image from the medical image management system 3 or the memory 21 in response to, for example, an operation made via the input interface 22. The medical image is, for example, three-dimensional volume data.

After step ST10, the processing circuitry 20 detects, at step ST20, anatomical positions from the acquired medical data. It is to be noted that, after separately acquiring, from a database (unillustrated) via the communication interface 23, the medical image on which all the ALDs corresponding to the anatomical positions are superimposed, the processing may advance to step ST30 by bypassing steps ST10 and ST20.

After step ST20, the processing circuitry 20 causes, at step ST30, the display 24 to display the medical image on which all the ALDs corresponding to the detected anatomical positions are superimposed. If step ST30 is performed after step ST60 or ST70, to be described below, the medical image subjected to the processing at step ST60 or ST70 is displayed. During the displaying of the medical image, the processing circuitry 20 may cause the display 24 to display, for example, an ALD display selection screen, which is a GUI for selectively narrowing down the anatomical landmarks to those to be displayed.

After step ST30, the processing circuitry 20 receives, at step ST40, a user's input operation made via the input interface 22. For example, the processing circuitry 20 receives an input operation on the ALD display selection screen 24a for selecting anatomical landmarks to be displayed relating to “bone”, with “trauma department” selected as the user's diagnosis and treatment department, as shown in FIG. 5.

After step ST40, the processing circuitry 20 determines, at step ST50, whether or not narrow-down information has been input by the received input operation, and, if narrow-down information has not been input, advances to step ST60. At step ST60, the processing circuitry 20 executes, according to the input contents, a separate process different from the process performed based on the narrow-down information.

If, on the other hand, narrow-down information is determined to have been input at step ST50, the processing circuitry 20 narrows down, at step ST70, based on the input narrow-down information, all the anatomical landmarks indicating the anatomical positions in the medical image to those to be displayed. Thereafter, the processing circuitry 20 causes the display 24 to display the medical image on which the narrowed-down anatomical landmarks are superimposed. It is assumed, for example, that, at step ST30 described above, the processing circuitry 20 displays, on the display 24, a medical image 24b on which anatomical landmarks ald relating to organs and bones are superimposed, as shown in FIG. 6(a). Based on the narrow-down information “trauma department” and “bone” input at step ST40, the processing circuitry 20 causes, at step ST50, the display 24 to display the medical image 24b on which narrowed-down anatomical landmarks ald relating to “trauma department” and “bone” are superimposed, as shown in FIG. 6(b). Since unnecessary anatomical landmarks ald are eliminated and the medical image 24b with necessary anatomical landmarks ald is displayed, it is possible to easily specify anatomical landmarks ald and other sites, thus improving the image reading efficiency.

After step ST60 or ST70, the processing circuitry 20 determines, at step ST80, whether or not to end the processing according to whether or not an exit instruction has been input, and, if the processing is determined not to be ended, returns to step ST30, and continues displaying a medical image. On the other hand, if the processing is determined to be ended at step ST80, the processing is ended.

According to the first embodiment described above, in the medical information processing apparatus 2, the processing circuitry 20 acquires a medical image. The processing circuitry 20 narrows down all the anatomical landmarks indicating anatomical positions in the medical image to those to be displayed. The processing circuitry 20 causes the display to display a medical image on which the narrowed-down anatomical landmarks are superimposed. With such a configuration of narrowing down the anatomical landmarks to those to be displayed, it is possible to display anatomical landmarks that do not interfere with reading of the medical image.

### Modification of First Embodiment

In the first embodiment, a case has been described as an example where narrow-down information specifying the diagnosis and treatment department and “bone” is input; however, the configuration is not limited thereto. Specifically, items that can be specified in the narrow-down information are not limited to the diagnosis and treatment department (trauma department) and the site (bone), and narrowing down may be performed by inputting narrow-down information specifying values of the items (e.g., “ALD classification”, “role”, “purpose”, “context”) contained in the association information 21a. For example, the narrow-down information may include a user's role. The processing circuitry 20 performs narrowing down in such a manner that at least predetermined anatomical landmarks are displayed if the narrow-down information contains the user's role and the user's role is a doctor-in-training. It is thereby possible to suppress narrowing down in such a manner that at least basic anatomical landmarks are not eliminated in the case of a doctor-in-training who is inexperienced, compared to an ordinary doctor.

### Second Embodiment

Next, a description will be given of a medical information processing apparatus according to a second embodiment.

In the second embodiment, which is a specific example of the first embodiment, narrowing down is performed in such a manner that anatomical landmarks of one or more sites to be preferentially read for each diagnosis and treatment department are displayed.

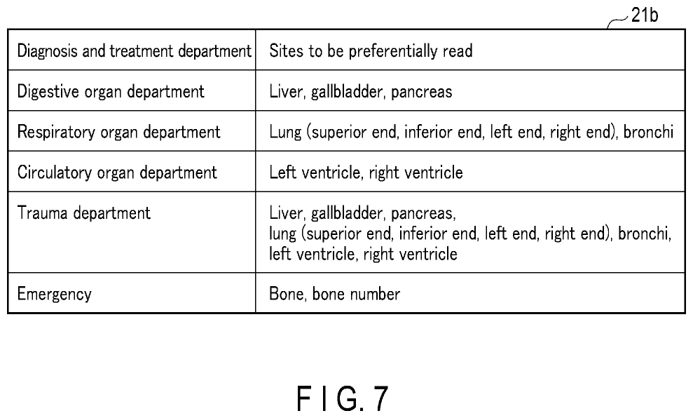

In accordance therewith, the memory 21 stores preferential site information 21b, as shown in FIG. 7. The preferential site information 21b is a table describing diagnosis and treatment departments and sites to be preferentially read in association with one another.

If the input narrow-down information contains a diagnosis and treatment department, the narrow-down function 203 of the processing circuitry 20 acquires, from the preferential site information 21b, one or more sites to be preferentially read based on the diagnosis and treatment department. Also, the narrow-down function 203 performs narrowing down in such a manner that anatomical landmarks relating to the acquired sites are displayed, by eliminating anatomical landmarks that are irrelevant to the acquired sites.

The other configurations are similar to those of the first embodiment.

With the above-described configuration, the processing circuitry 20 acquires, from the preferential site information 21b, one or more sites to be preferentially read based on the diagnosis and treatment department in the input narrow-down information, and performs narrowing down in such a manner that anatomical landmarks relating to the acquired sites are displayed. Similarly to the above-described configuration, the processing circuitry 20 causes the display to display a medical image on which narrowed-down anatomical landmarks are superimposed.

Thus, according to the second embodiment, with the configuration of using the preferential site information 21b, it is possible, in addition to the above-described effects, to easily associate a diagnosis and treatment department and sites to be preferentially read. In addition, with the configuration of using the preferential site information 21b, it is possible to perform narrowing down in such a manner that anatomical landmarks relating to sites to be preferentially read are displayed according to the diagnosis and treatment department.

### Third Embodiment

Next, a description will be given of a medical information processing apparatus according to a third embodiment.

In the third embodiment, which is a modification of the first and second embodiments, enlargement is performed as the separate process at step ST60.

In accordance therewith, the narrow-down function 203 of the processing circuitry 20 performs, in addition to the above-described function, narrowing down in such a manner that the number of anatomical landmarks to be displayed is increased in response to the input enlargement instruction.

The display control function 204 causes, in addition to the above-described function, the display 24 to enlarge the medical image based on the enlargement instruction.

The other configurations are similar to those of the first and second embodiments.

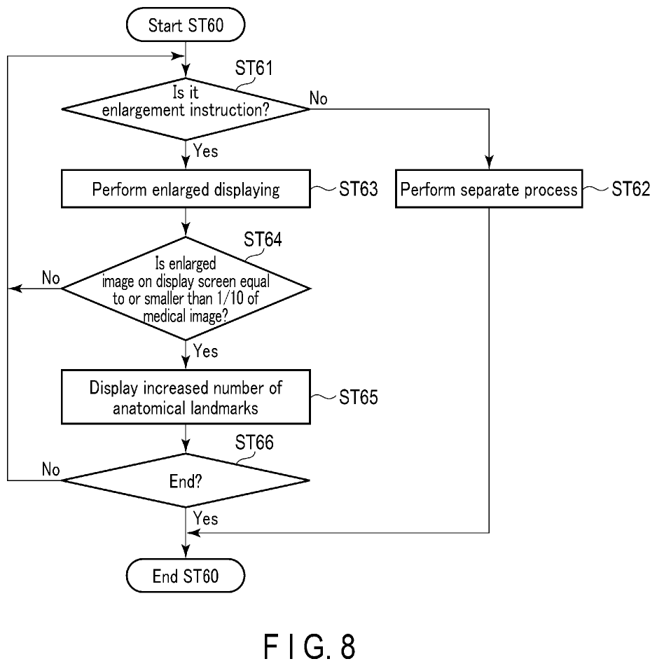

Next, an operation of the medical information processing apparatus with the above-described configuration will be described with reference to the flowchart of FIG. 8.

It is assumed that, after steps ST10 to ST80 are executed, a medical image on which narrowed-down anatomical landmarks are superimposed is being displayed on the display 24 at step ST30 performed a second time. At step ST40, the processing circuitry 20 receives a user's input operation made via the input interface 22. It is assumed, for example, that the processing circuitry 20 has received an input operation for an enlargement instruction to enlarge the medical image being displayed.

After step ST40, the processing circuitry 20 determines, at step ST50, whether or not narrow-down information has been input by the received input operation. Since the enlargement instruction has been input, the processing circuitry 20 determines that the narrow-down information has not been input, and advances to step ST60, as shown in FIG. 8.

At step ST60, the processing circuitry 20 executes a separate process different from the process relating to the narrow-down information. The separate process includes steps ST61 to ST66.

At step ST61, the processing circuitry 20 determines whether or not an enlargement instruction has been input by the received input operation, and, if a narrow-down instruction has not been input, advances to step ST62. At step ST62, the processing circuitry 20 executes, according to the input contents, a separate process different from the enlargement process.

On the other hand, if an enlargement instruction is determined to have been input at step ST61, the processing circuitry 20 causes, at step ST63, the display 24 to enlarge a medical image on which anatomical landmarks are superimposed in response to the input enlargement instruction. In the enlarged medical image, an interval between the anatomical landmarks is increased, and the number of displayed anatomical landmarks is decreased by the number of anatomical landmarks located outside the display screen, compared to a non-enlarged medical image.

After step ST63, the processing circuitry 20 determines, at step ST64, whether or not the enlarged medical image on the display screen is equal to or smaller than 1/10 of the original medical image, and returns to step ST61 if it is not. Thereby, an enlarging process of the display screen is repeatedly executed by a loop of steps ST61, ST63, and ST64. It is to be noted that the threshold value “ 1/10” is given as an example, and the configuration is not limited thereto.

On the other hand, it is assumed that the enlarged medical image on the display screen is determined, at step ST64, to have become equal to or smaller than 1/10 of the original medical image. In this case, the processing circuitry 20 performs, at step ST65, narrowing down to increase the number of anatomical landmarks to be displayed. Thereafter, the processing circuitry 20 causes the display 24 to display the medical image on which the narrowed-down anatomical landmarks are superimposed. With the enlargement, since new anatomical landmarks ald are displayed between a plurality of anatomical landmarks ald located at an increased interval, it is possible to avoid a situation in which the number of anatomical landmarks is reduced to zero by an enlarging process, thereby maintaining the image reading efficiency.

After step ST62 or ST65, the processing circuitry 20 determines, at step ST66, whether or not to end the processing according to whether or not an exit instruction has been input, and, if the processing is determined not to be ended, returns to step ST61 and continues the processing. On the other hand, if the processing is determined to be ended at step ST66, the processing at step ST60 is ended, and shifts to step ST80.

According to the third embodiment described above, the processing circuitry 20 performs narrowing down in such a manner that the number of anatomical landmarks to be displayed increases in response to the input enlargement instruction. Also, the processing circuitry 20 causes the display 24 to enlarge the medical image based on the enlargement instruction.

Therefore, it is possible to suppress a decrease in image reading efficiency caused by a decrease in the number of anatomical landmarks as a result of the enlargement.

### Fourth Embodiment

Next, a description will be given of a medical information processing apparatus according to a fourth embodiment.

In the fourth embodiment, which is a modification of the first to third embodiments, narrowing down is performed in such a manner that anatomical landmarks are displayed in accordance with contents selected to be displayed for each user ID, which is an identifier assigned to the user.

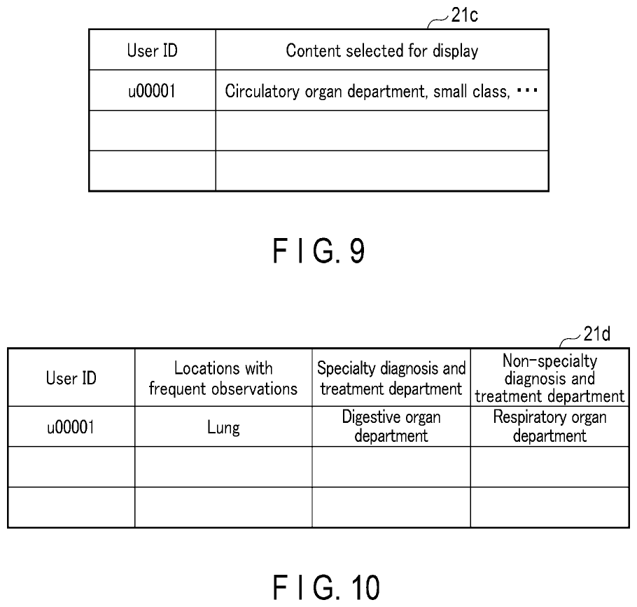

In accordance therewith, the memory 21 stores history information 21c, as shown in FIG. 9. The history information 21c is a table describing user IDs and contents selected to be displayed, which are narrow-down information that has been input in the past, in association with one another. The memory 21 and the history information 21c are examples of a first memory configured to store narrow-down information by the identifier assigned to the user.

Upon inputting of a user ID, the narrow-down function 203 of the processing circuitry 20 reads contents selected to be displayed (narrow-down information) from the history information 21c in the memory 21 based on the user ID, and performs narrowing down in such a manner that anatomical landmarks are displayed according to the read contents selected to be displayed (narrow-down information).

The other configurations are similar to those of the first to third embodiments.

With the above-described configuration, the processing circuitry 20 performs, based on the input user ID, narrowing down in such a manner that anatomical landmarks are displayed according to the contents selected to be displayed (narrow-down information) read from the history information 21c in the memory 21. Similarly to the above-described configuration, the processing circuitry 20 causes the display to display a medical image on which narrowed-down anatomical landmarks are superimposed.

Thus, according to the fourth embodiment, with the configuration of using the history information 21c, it is possible, in addition to the above-described effects, to easily associate a user ID with past contents selected to be displayed. With the configuration of using the history information 21c, it is possible to perform narrowing down in such a manner that anatomical landmarks relating to contents selected to be displayed are displayed according to the user ID. That is, since a site that is difficult to read (unfamiliar for reading) differs according to the user, a site for which anatomical landmarks should be displayed differs according to the user. With the fourth embodiment, it is possible to display anatomical landmarks as desired by each user, since anatomical landmarks can be narrowed down according to past contents selected to be displayed for each user ID.

### Fifth Embodiment

Next, a description will be given of a medical information processing apparatus according to a fifth embodiment.

In the fifth embodiment, which is a modification of the first to third embodiments, narrowing down is performed in such a manner that anatomical landmarks are displayed for each user ID according to the diagnosis and treatment department, etc. relating to the user's image reading.

In accordance therewith, the memory 21 stores user assistance information 21d as shown in FIG. 10. The user assistance information 21d is a table describing, in association with one another, user IDs, locations for which observations are often made (sites for which observations have often been made in the past), specialty diagnosis and treatment departments, and non-specialty diagnosis and treatment departments. It is to be noted that the locations for which observations are often made may be described in the user assistance information 21d by the processing circuitry 20 based on, for example, a prior image reading report. The memory 21 and the user assistance information 21d are examples of a second memory configured to store, for each identifier assigned to the user, a diagnosis and treatment department relating to the user's image reading.

The narrow-down function 203 of the processing circuitry 20 performs narrowing down in such a manner that anatomical landmarks corresponding to the diagnosis and treatment department in the user assistance information 21d stored in the memory 21 are displayed based on the input user ID.

The other configurations are similar to those of the first to third embodiments.

With the above-described configuration, the processing circuitry 20 performs narrowing down in such a manner that anatomical landmarks are displayed according to the diagnosis and treatment department in the user assistance information 21d stored in the memory 21 based on the input user ID. Similarly to the above-described configuration, the processing circuitry 20 causes the display to display a medical image on which narrowed-down anatomical landmarks are superimposed.

Thus, according to the fifth embodiment, with the configuration of using the user assistance information 21d, it is possible, in addition to the above-described effects, to easily associate a user ID with a diagnosis and treatment department relating to the user's image reading. With the configuration of using the user assistance information 21d, it is possible to perform narrowing down in such a manner that anatomical landmarks relating to image reading are displayed according to the user ID. That is, since a site relating to image reading differs according to the user, a site for which anatomical landmarks should be displayed differs according to the user. With the fifth embodiment, it is possible to display, for each user, anatomical landmarks relating to image reading, since anatomical landmarks are narrowed down according to the diagnosis and treatment department relating to image reading of each user ID.

### Sixth Embodiment

Next, a description will be given of a medical information processing apparatus according to a sixth embodiment.

In the sixth embodiment, which is a modification of the first to fifth embodiments, narrowing down is performed in such a manner that anatomical landmarks located within a predetermined range from a cursor displayed on a medical image are displayed. It is to be noted that anatomical landmarks may be displayed in the vicinity of the measurement points, as well as a cursor.

In accordance therewith, if a cursor or measurement points are displayed on the medical image, the narrow-down function 203 of the processing circuitry 20 performs, in addition to the above-described function, narrowing down in such a manner that anatomical landmarks located within a predetermined range from the displayed cursor or measurement points are displayed.

The other configurations are similar to those of the first to fifth embodiments.

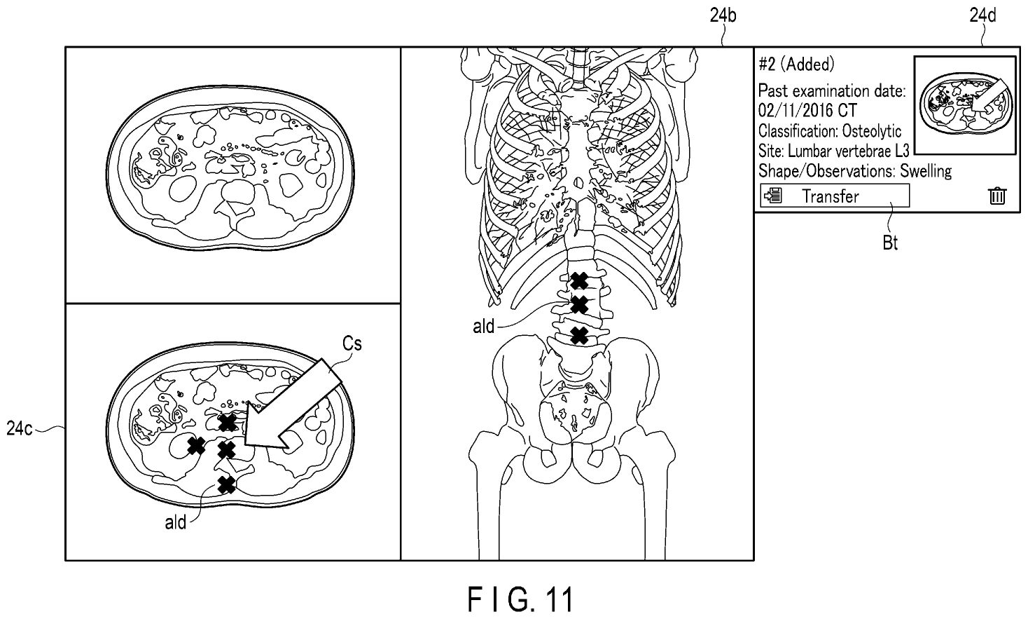

With the above-described configuration, if a cursor Cs is displayed on the medical image 24c, as shown in FIG. 11, the processing circuitry 20 performs narrowing down in such a manner that anatomical landmarks ald located within a predetermined range from the displayed cursor Cs are displayed. Also, similarly to the above-described configuration, the processing circuitry 20 causes the display 24 to display a medical image 24c on which narrowed-down anatomical landmarks ald are superimposed.

Moreover, the processing circuitry 20 may suitably display, in parallel on the display 24, a medical image 24c indicating a cross-section of a subject including the lumbar vertebrae, and a medical image 24b indicating a skeleton of the front of the abdomen of the subject corresponding to the medical image 24c. Furthermore, the processing circuitry 20 may cause the display 24 to further display an observation attachment screen 24d for attaching a medical image 24c to an image reading report. The observation attachment screen may include, for example, a thumbnail image of the medical image 24c, a description to be added to the image reading report (e.g., a date of examination, a classification, a site, a shape, and observations), and a transfer button Bt for executing the attaching.

Thus, according to the sixth embodiment, if a cursor Cs is displayed on the medical image 24c, the processing circuitry 20 performs narrowing down in such a manner that anatomical landmarks ald located within a predetermined range from the displayed cursor Cs are displayed. With the configuration of displaying anatomical landmarks in the vicinity of a region directly instructed by the cursor, it is possible, in addition to the above-described effects, to display anatomical landmarks in the vicinity of a desired site more directly.

### Modification of Sixth Embodiment

In the sixth embodiment, anatomical landmarks ald are displayed in the vicinity of the cursor Cs; however, the configuration is not limited thereto. For example, anatomical landmarks ald may be displayed in the vicinity of measurement points, in place of the cursor Cs. In this case, if measurement points are displayed on the medical image 24c, the processing circuitry 20 performs narrowing down in such a manner that anatomical landmarks ald located within a predetermined range from the displayed measurement points are displayed. In the case of, for example, calculating a cardiothoracic ratio from the medical image, measurement points for detecting, from a frontal chest X-ray image, a cardiac silhouette and a thoracic silhouette, and measuring a cardiac width and a thoracic width from the respective silhouettes can be employed. It is to be noted that the measurement points may be derived by an image processing algorithm of the image processing function 202 on the medical image, or may be specified on the medical image by a user operation. In either case, it is possible to obtain similar effects as can be obtained by the sixth embodiment.

### Seventh Embodiment

Next, a description will be given of a medical information processing apparatus according to a seventh embodiment.

In the seventh embodiment, which is a modification of the first to sixth embodiments, a lesion displaying process is performed as the separate process at step ST60.

In accordance therewith, upon detecting a lesion in a medical image, the narrow-down function 203 of the processing circuitry 20 performs, in addition to the above-described function, narrowing down in such a manner that anatomical landmarks located within a predetermined range from a position of the lesion are displayed.

The other configurations are similar to those of the first to sixth embodiments.

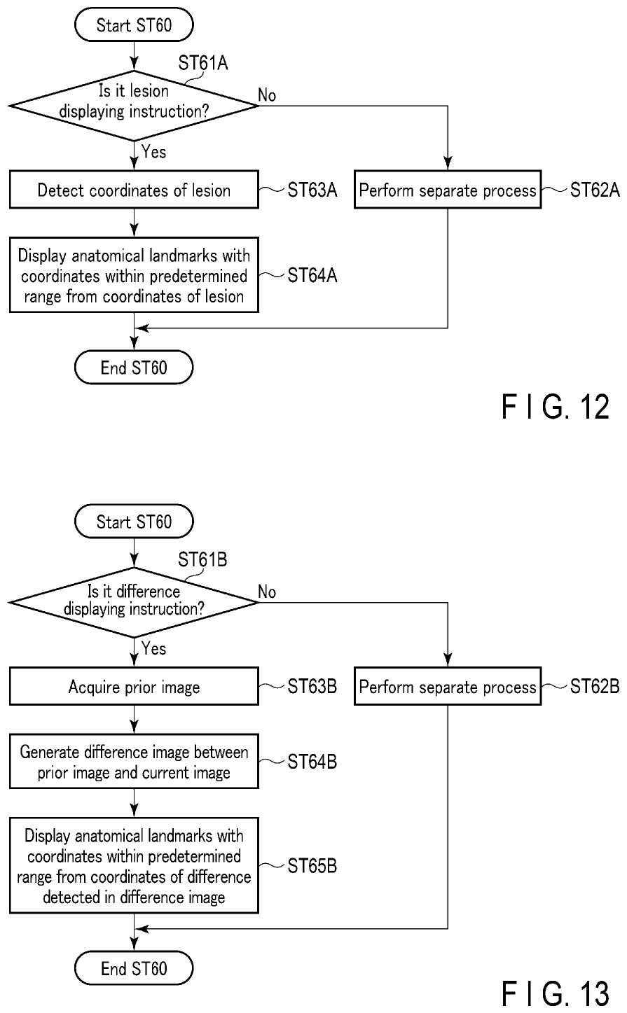

Next, an operation of the medical information processing apparatus with the above-described configuration will be described with reference to the flowchart of FIG. 12.

It is assumed that, after steps ST10 to ST80 are executed, a medical image on which narrowed-down anatomical landmarks are superimposed is being displayed on the display 24 at step ST30 performed a second time. At step ST40, the processing circuitry 20 receives a user's input operation made via the input interface 22. It is assumed, for example, that the processing circuitry 20 has received an input operation a lesion displaying instruction to display a lesion in a medical image being displayed.

After step ST40, the processing circuitry 20 determines, at step ST50, whether or not narrow-down information has been input by the received input operation. Since the lesion displaying instruction has been input, the lesion displaying instruction processing circuitry 20 determines that the narrow-down information has not been input, and advances to step ST60, as shown in FIG. 12.

At step ST60, the processing circuitry 20 executes a separate process different from the process relating to the narrow-down information. The separate process includes steps ST61A to ST64A.

At step ST61A, the processing circuitry 20 determines whether or not a lesion displaying instruction has been input by the received input operation, and, if a lesion displaying instruction has not been input, advances to step ST62A. At step ST62A, the processing circuitry 20 executes, according to the input contents, a separate process different from the lesion displaying process.

On the other hand, if a lesion displaying instruction is determined to have been input at step ST61A, the processing circuitry 20 detects, at step ST63A, coordinates of a lesion from a medical image on which anatomical landmarks are superimposed, in response to the input lesion displaying instruction. It is to be noted that, at step ST63A, coordinates of a lesion detected in advance by CAD may be read from incidental information of the medical image, or a lesion and its coordinates may be newly detected from the medical image.

After step ST63A, the processing circuitry 20 performs, at step ST64A, narrowing down in such a manner that anatomical landmarks ald located within a predetermined range from the coordinates of the lesion are displayed. Also, similarly to the above-described configuration, the processing circuitry 20 causes the display 24 to display a medical image on which narrowed-down anatomical landmarks ald are superimposed.

After step ST62A or ST64A, the processing circuitry 20 ends step ST60 and shifts to step ST80.

According to the seventh embodiment described above, upon detecting a lesion in the medical image, the processing circuitry 20 performs narrowing down in such a manner that anatomical landmarks located within a predetermined range from a position of the lesion are displayed. Accordingly, it is possible, in addition to the above-described effects, to easily specify a site in the vicinity of the lesion in the medical image at the time of reading the lesion.

### Eighth Embodiment

Next, a description will be given of a medical information processing apparatus according to an eighth embodiment.

In the eighth embodiment, which is a modification of the first to seventh embodiments, a difference displaying process is performed as the separate process at step ST60.

In accordance therewith, the image processing function 202 of the processing circuitry 20 is capable of generating, in addition to the above-described function, a difference image indicating one or more differences between a medical image and a past medical image corresponding to the medical image.

Upon detecting a difference between the medical image and the past medical image corresponding to the medical image, the narrow-down function 203 performs, in addition to the above-described function, narrowing down in such a manner that anatomical landmarks located within a predetermined range from a position of the difference are displayed. It is to be noted that the past medical image is a prior image obtained by imaging a site of a subject of a medical image in the past. That is, the medical image and the past medical image are images for observing changes over time of the same site of the same subject. Accordingly, a medical image may be referred to as a “current image”, and the past medical image may be referred to as a “prior image”.

The other configurations are similar to those of the first to seventh embodiments.

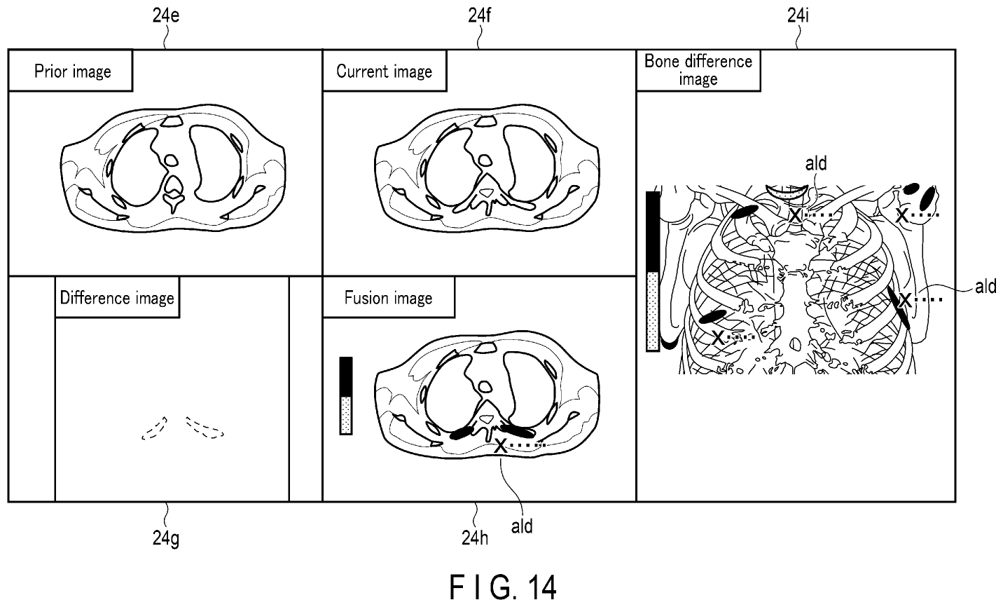

Next, an operation of the X-ray diagnostic apparatus with the above-described configuration will be described with reference to the flowchart of FIG. 13 and the schematic diagram of FIG. 14.

It is assumed that, after steps ST10 to ST80 are executed, a medical image (current image) on which narrowed-down anatomical landmarks are superimposed is being displayed on the display 24 at step ST30 performed a second time. At step ST40, the processing circuitry 20 receives a user's input operation made via the input interface 22. It is assumed, for example, that the processing circuitry 20 has received an input operation for a difference displaying instruction to display one or more differences between the current image being displayed and a prior image (past medical image) corresponding thereto.

After step ST40, the processing circuitry 20 determines, at step ST50, whether or not narrow-down information has been input by the received input operation. Since the difference displaying instruction has been input, the processing circuitry 20 determines that the narrow-down information has not been input, and advances to step ST60, as shown in FIG. 13.

At step ST60, the processing circuitry 20 executes a separate process different from the process relating to the narrow-down information. The separate process includes steps ST61B to ST65B.

At step ST61B, the processing circuitry 20 determines whether or not a difference displaying instruction has been input by the received input operation, and, if a difference displaying instruction has not been input, advances to step ST62B. At step ST62B, the processing circuitry 20 executes, according to the input contents, a separate process different from the difference displaying process.

On the other hand, if a difference displaying instruction is determined to have been input at step ST61B, the processing circuitry 20 acquires, at step ST63B, a prior image corresponding to the current image being displayed from the medical image management system 3, in response to the input difference displaying instruction. Also, the processing circuitry 20 displays the prior image 24e side by side with the current image 24f, as shown in FIG. 14.

After step ST63B, the processing circuitry 20 detects, at step ST64B, a difference between the prior image 24e and the current image 24f, generates a difference image 24g indicating the difference, and causes the display 24 to display the generated difference image 24g. The processing circuitry 20 performs, for example, a process of emphasizing a bone region of each of the prior image 24e and the current image 24f, and performs a linear positioning process between the prior image 24e and the current image 24f in which the bone region is emphasized. After the linear positioning process, a non-linear positioning process is performed using, for example, an LDDMM technique. “LDDMM” stands for “large deformation diffeomorphic metric mapping”. Thereafter, a bone emphasizing process is performed after the non-linear positioning process. The bone emphasizing process is a process of maintaining only one or more differences relating to bone at the time of difference calculation. After the bone emphasizing process, an adaptive differential process such as Adaptive Voxel Matching is performed. Thereby, the processing circuitry 20 generates a difference image 24g in which only changes in bone characteristics between the prior image 24e and the current image 24f are emphasized. It is to be noted that the process of generating the difference image 24g is not limited to the above-described one, and a given approach may be employed.

After step ST64B, upon detecting a difference between the current image 24f and a prior image 24e corresponding to the current image 24f, the processing circuitry 20 performs, at step ST65B, narrowing down in such a manner that anatomical landmarks located within a predetermined range from a position of the difference are displayed. Also, the processing circuitry 20 causes the display 24 to display a current image on which the narrowed-down anatomical landmarks ald are superimposed and the difference is emphasized as a fusion image 24h. However, the fusion image 24h (emphasized displaying of the difference) need not be used, and a current image 24f on which narrowed-down anatomical landmarks ald are superimposed may be displayed. Also, the processing circuitry 20 may cause the display 24 to suitably display a bone difference image 24i, which is a three-dimensional image indicating a frontal skeleton of the subject corresponding to the fusion image 24h. It is to be noted that, in the bone difference image 24i, a shaded portion in the vicinity of the cervical vertebrae shows a bone decreasing portion (a minus difference) for emphasis. In the bone difference image 24i, the blacked-out portions show bone increasing portions (plus differences) for emphasis. The bone difference image 24i may be referred to as a “fusion 3D image”.

After step ST62B or ST65B, the processing circuitry 20 ends step ST60, and shifts to step ST80.

According to the eighth embodiment described above, upon detecting a difference between the medical image and the past medical image corresponding to the medical image, the processing circuitry 20 performs narrowing down in such a manner that anatomical landmarks located within a predetermined range from a position of the difference are displayed. Accordingly, at the time of reading a portion that has changed over time in the medical image, it is possible, in addition to the above-described effects, to easily specify a site in the vicinity of the changed portion.

In addition, the functions according to the embodiment may be implemented by installing programs for executing the processes according to the present embodiment into a computer such as a workstation, and expanding the programs on the memory. At this time, the programs capable of causing the computer to execute the processes may be stored in a storage medium such as a magnetic disk (hard disk), an optical disk (a CD-ROM, a DVD, a Blu-ray (registered trademark) etc.), a semiconductor memory, etc. and distributed.

According to at least one of the above-described embodiments, it is possible to display anatomical landmarks so as not to interfere with reading of the medical image.

The term “processor” used herein refers to, for example, a CPU or a GPU, or various types of circuitry, such as an application-specific integrated circuit (ASIC), a programmable logic device (e.g., a simple programmable logic device (SPLD), a complex programmable logic device (CPLD), or a field programmable gate array (FPGA)), etc. If the processor is, for example, a CPU, the processor reads and executes programs stored in a memory to realize the functions. If the processor is, for example, an ASIC, the functions are directly incorporated into the circuitry of the processor as logic circuitry, instead of the programs being stored in the memory. Each processor in the present embodiment is not limited to a single circuitry-type processor, and multiple independent circuits may be combined and integrated as a single processor to realize the intended functions. Furthermore, multiple components or features as given in FIG. 1 may be integrated as one processor to realize the respective functions.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing apparatus comprising processing circuitry configured to:
acquire a medical image;
narrow down a plurality of anatomical landmarks each indicating an anatomical position in the medical image to one or more anatomical landmarks to be displayed; and
cause a display to display the medical image on which the one or more narrowed-down anatomical landmarks are superimposed.

2. The medical information processing apparatus according to claim 1, wherein
the processing circuitry is configured to perform the narrowing down based on narrow-down information that has been input.

3. The medical information processing apparatus according to claim 2, wherein
the narrow-down information contains a role of a user, and
the processing circuitry is configured to perform the narrowing down in such a manner that at least one or more predetermined anatomical landmarks are displayed if the narrow-down information contains the role of the user and the role of the user is a doctor-in-training.

4. The medical information processing apparatus according to claim 2, further comprising:
a first memory configured to store the narrow-down information by an identifier of the user, wherein
the processing circuitry is configured to perform the narrowing down in such a manner that, if the identifier is input, the narrow-down information is read from the first memory based on the identifier, and one or more anatomical landmarks corresponding to the read narrow-down information are displayed.

5. The medical information processing apparatus according to claim 1, further comprising:
a second memory configured to store, by an identifier of the user, a diagnosis and treatment department relating to image reading by the user, wherein
the processing circuitry is configured to perform the narrowing down in such a manner that one or more anatomical landmarks corresponding to the diagnosis and treatment department in the second memory are displayed based on the input identifier.

6. The medical information processing apparatus according to claim 1, wherein
the processing circuitry is configured to:
perform, in response to inputting of an enlargement instruction, the narrowing down in such a manner that the anatomical landmarks to be displayed are increased in number; and
cause the display to perform enlarge the medical image based on the enlargement instruction.

7. The medical information processing apparatus according to claim 1, wherein
the processing circuitry is configured to perform, if a cursor or at least one measurement point is displayed on the medical image, the narrowing down in such a manner that one or more anatomical landmarks located within a predetermined range from the displayed cursor or measurement point are displayed.

8. The medical information processing apparatus according to claim 1, wherein
the processing circuitry is configured to perform, if a lesion is detected in the medical image, the narrowing down in such a manner that one or more anatomical landmarks located within a predetermined range from a position of the lesion are displayed.

9. The medical information processing apparatus according to claim 1, wherein
the processing circuitry is configured to perform, if a difference is detected between the medical image and a past medical image corresponding to the medical image, the narrowing down in such a manner that one or more anatomical landmarks located within a predetermined range from a position of the difference are displayed.

10. A medical information processing method comprising:
acquiring a medical image;
narrowing down a plurality of anatomical landmarks each indicating an anatomical position in the medical image to one or more anatomical landmarks to be displayed; and
causing a display to display the medical image on which the one or more narrowed-down anatomical landmarks are superimposed.
